(19) United States
(12) Patent Application Publication
(10) Pub. No.: US 2020/0121695 A1
(43) Pub. Date: Apr. 23, 2020

(54) COMPOSITIONS AND METHODS FOR TREATING ITCH, SKIN INFLAMMATION, AND PRURITUS

(71) Applicant: Jill Renov BLISKO, Lawrence, NY (US)

(72) Inventor: Jill Renov BLISKO, Lawrence, NY (US)

(21) Appl. No.: 16/622,203

(22) PCT Filed: Jun. 12, 2018
(86) PCT No.: PCT/US18/37104

§ 371 Date: Dec. 12, 2019

Related U.S. Application Data
(60) Provisional application No. 62/518,543, filed on Jun. 12, 2017.

(51) Int. Cl. A61K 31/575; A61K 31/5377; A61P 17/04; A61P 29/00; A61K 45/06; G01N 33/573; G01N 33/74

(52) U.S. Cl. CPC A61K 31/575; A61K 31/5377; A61P 17/04; G01N 33/743; A61K 45/06; G01N 33/573; A61P 29/00

(57) ABSTRACT

The invention provides compositions and methods for treating an itch, a skin inflammation, or a pruritus. Specifically, the invention relates to the use of a progesterone antagonist, an agent that antagonizes the elevated level of alkaline phosphatase (ALP), or a combination thereof to treat an itch, a skin inflammation, or a pruritus.

### CROSS-REFERENCE TO RELATED APPLICATIONS

This application claims priority to and the benefit of U.S. Provisional Patent Application 62/518,543, filed Jun. 12, 2017, which is incorporated by reference herein in its entirety.

### FIELD OF THE INVENTION

The invention relates to compositions and methods for treating an itch, a skin inflammation, or a pruritus. Specifically, the invention relates to the use of a progesterone antagonist, an agent that antagonizes the elevated level of alkaline phosphatase (ALP), or a combination thereof for treating an itch, a skin inflammation, or a pruritus.

### BACKGROUND OF THE INVENTION

Pruritus is a physiological perception within the sensory neuronal network in the skin which, along with pain and physical or mechanical stimuli, can serve as a warning system against potential bodily threats. Itching is an unpleasant sensation that can lead to scratching, but is independent of pain. The International Federation for the Study of Itch (IFSI) defines chronic pruritus (as opposed to acute pruritus) as itching that lasting six weeks or longer (S. Stander et al., Acta Derm. Venereol., 2007, 87(4):291-4). Several factors in and on the skin can activate the sensory nerve fibers or modulate their activity and thus trigger, suppress, or exacerbate itching. Physical stimuli such as cold and heat modulate the perception of itching; painful heat and cold can significantly diminish it, while moderate cold intensifies it (Valet et al., J. Invest. Dermatol., 2008, 128(2):426-33.). Mechanical factors such as rubbing or scratching the skin can briefly suppress itching by activating nerve fibers that selectively activate or de-activate certain areas of the brain (Yosipovitch et al., J. Invest. Dermatol., 2008, 128(7):1806-11).

Chronic pruritus can seriously diminish the quality of life in its sufferers. It is a seriously debilitating condition, comparable to chronic pain, which can lead to frustration, desperation and depression. Moreover, chronic scratching often produces open skin lesions, subject to primary or secondary infection, scarring and potential disfigurement. Chronic pruritus is often an indication of underlying disease. Diagnosis of the underlying disease is desirable, and clinical presentation, patient history, and patient self-evaluation form important parts of such diagnosis.

Chronic pruritus may be observed in patients with inflammatory skin disease including, but not limited to, atopic dermatitis, allergic, irritant contact dermatitis, exsiccation dermatitis, nummular and dyshidrotic dermatitis, lichen planus, lichen sclerosus et atrophicus, polymorphous light eruption psoriasis, Grover's disease, mucinosis, mastocytosis, and urticaria; infectious skin diseases such as mycoses, bacterial and viral infections, scabies, pediculosis, insect bites, and folliculitides; autoimmune skin diseases including, but not limited to, Bullous skin disorders, especially dermatitis herpetiformis (Duhring's disease), and bullous pemphigoid; genodermatoses such as Darier's disease, and Hailey-Hailey disease; pregnancy-related skin diseases including, but not limited to, polymorphic eruption of pregnancy (PEP, also known as PUPPP), atopic eruption of pregnancy, and pemphigoid gestationis; and neoplasias such as cutaneous T-cell lymphoma (especially the erythrodermic form).

Elevated alkaline phosphatase describes the situation where the levels of alkaline phosphatase (ALP) exceed the reference range (i.e., the normal range). This group of enzymes catalyzes the hydrolysis of phosphate esters in an acidic environment. The major function of alkaline phosphatase is transporting across cell membranes. Alkaline phosphatases are present in many human tissues, including bone, intestine, kidney, liver, placenta and white blood cells. Damage to these tissues causes the release of ALP into the bloodstream. Elevated alkaline phosphate is associated with certain medical conditions or syndromes. It serves as a significant indication for certain medical conditions, diseases and syndromes. Elevated alkaline phosphatase in liver is indicative of multiple pathological conditions, among which are primary biliary cirrhosis and autoimmune hepatitis. Other conditions that may lead to elevated liver ALP are, without limitation, cholestasis, cholecystitis, cholangitis, cirrhosis, fatty liver, sarcoidosis, liver tumor, liver metastases, or drugs (e.g. verapamil, carbamazepine, phenytoin, erythromycin, allopurinol, ranitidine).

Primary biliary cirrhosis, (PBC), is a chronic disease of the liver that slowly destroys the medium-sized bile ducts within the liver. In patients with PBC, the bile ducts are destroyed by inflammation. This causes bile to remain in the liver, where gradual injury damages liver cells and causes cirrhosis, or scarring of the liver. As cirrhosis progresses and the amount of scar tissue in the liver increases, the liver loses its ability to function. Cirrhosis also prevents blood from the intestines from returning to the heart. Many people with PBC do not have symptoms, especially in the early stages of the disease, and PBC is typically not diagnosed before 35 and even more commonly not before 45 years of age. When symptoms do occur the most common is pruritus, or intense itching of the skin, often in the arms, legs and back. Other symptoms may include fatigue, jaundice, fluid build-up in the ankles and abdomen, and darkening of the skin and collection of fatty deposits in the skin around the eye. PBC can eventually progress to cirrhosis of the liver. This in turn may lead to a number of symptoms or complications such as fluid retention in the abdomen (ascites), enlarged spleen, oesophageal varices, hepatic encephalopathy, including coma in extreme cases.

Autoimmune hepatitis (AIH) is a generally unresolving inflammation of the liver of unknown cause. A working model for its pathogenesis postulates that environmental triggers, a failure of immune tolerance mechanisms, and a genetic predisposition collaborate to induce a T cell-mediated immune attack upon liver antigens, leading to a progressive necroinflammatory and fibrotic process in the liver. Onset is frequently insidious with nonspecific symptoms such as fatigue, muscle aches jaundice, nausea, abdominal pain, and arthralgias at presentation but the clinical spectrum is wide, ranging from an asymptomatic presentation to an acute severe disease. The disease usually affects women and is strongly associated with anti-smooth muscle autoantibodies.

Autoimmune progesterone dermatitis (APD) is a rare disorder characterized by recurrent skin manifestations starting during the luteal phase of a woman's menstrual cycle. The rash generally appears during the second half of the cycle when levels of the hormone, progesterone, begin to rise and it subsides shortly after menstruation. Although the exact underlying cause of APD is not well understood, it is thought to involve an abnormal immune reaction (autoimmune response) triggered by a woman's own progesterone. One proposed mechanism is sensitization to one's excess progesterone that occurs in the course of using exogenous progesterone, e.g. oral hormonal birth control pills. Thus risk factors include fertile women with a previous history of exogenous progesterone intake and sometimes with pregnancy. Signs and symptoms of APD can present as urticaria, angioedema, erythema multiforme, and eczematous lesions, thought to be an unusual allergy to progesterone. These skin issues can appear around mouth, on lips, upper body, legs, arms (elbows), palms, hands, or feet.

Pruritic urticarial papules and plaques of pregnancy (PUPPP) is a chronic hives-like rash that strikes some women during pregnancy. The cause of the condition is generally unknown. PUPPP frequently begins on the abdomen and spreads to the legs, feet, arms, and chest. Papules and plaques usually start appearing on the abdomen (although not on the umbilicus/bellybutton) and often spreads to the legs, chest, underarms, etc. Skin distension (stretching) is a common factor in PUPPP, which is more common in mothers with large fundal measurements and/or those who are carrying large babies, twins, and triplets. The papules and plaques often first appear within stretch marks. This skin condition occurs mostly in first pregnancies (primigravida), in the third trimester and is more likely with multiples in pregnancies.

APD and PUPPP affect mainly fertile women of child-bearing age, whereas PBC and AIH are generally diagnosed at a later age, at least at the stage of pre-menopausal, if not already at a later stage of menopause characterized by lower overall progesterone levels. There are few patients where these conditions are thought to overlap. Originally, it was thought that the two sets of conditions are wholly unrelated. However, since PBC and AIH may be asymptomatic for extended periods of time, there may be in fact a causal connection between these liver autoimmune disorders and progesterone-autoimmunity driven pathologies. In addition, the available PUPPP treatments focus on exclusively symptomatic relief while APD treatments may also involve temporary suppression of ovulation (e.g., by Lupron), which in turn may prompt symptoms of menopause. Thus, there exists a need for compositions and methods for treating itch, skin inflammation, or pruritus associated diseases and conditions.

### SUMMARY OF THE INVENTION

In one aspect, the present invention provides a method for treating an itch, a skin inflammation, or a pruritus in a subject having elevated liver alkaline phosphatase (ALP), and further having elevated progesterone, the method comprising: administering to said subject a pharmaceutical composition comprising therapeutically effective amount of a progesterone antagonist, an agent that antagonizes elevated ALP, or a combination thereof.

In another aspect, the present invention provides a method for treating an itch, a skin inflammation, or a pruritus in a subject having elevated liver ALP, the method comprising: administering to said subject a pharmaceutical composition comprising therapeutically effective amount of a progesterone antagonist, an agent that antagonizes elevated ALP, or a combination thereof.

In yet another aspect, the present invention provides a method for treating an itch, a skin inflammation, or a pruritus in a subject having elevated progesterone, the method comprising: administering to said subject a pharmaceutical composition comprising therapeutically effective amount of a progesterone antagonist, an agent that antagonizes elevated ALP, or a combination thereof.

In a further aspect, the present invention provides a pharmaceutical composition comprising a progesterone antagonist, an agent that antagonizes elevated ALP, or a combination thereof, wherein said antagonist, agent, or both are present in said composition in an amount effective to treat an itch, a skin inflammation, or a pruritus in a subject having elevated liver alkaline phosphatase (ALP), and further having elevated progesterone.

In a further aspect, the present invention provides a pharmaceutical composition comprising an agent that antagonizes elevated ALP, a progesterone antagonist, or a combination thereof, wherein said antagonist, agent, or both are present in said composition in an amount effective to treat an itch, a skin inflammation, or a pruritus in a subject having elevated liver ALP.

In an additional aspect, the present invention provides a pharmaceutical composition comprising an agent that antagonizes elevated ALP, a progesterone antagonist, or a combination thereof—wherein said antagonist, agent, or both are present in said composition in an amount effective to treat an itch, a skin inflammation, or a pruritus in a subject having elevated progesterone.

In an additional aspect, the present invention provides a method for preventing an itch, a skin inflammation, or a pruritus in a subject, the method comprising: determining the level of liver alkaline phosphatase (ALP); determining the level of progesterone; and administering a progesterone inhibitor to the subject if the level of ALP or the level of progesterone exceeds a threshold level, thereby preventing an itch, a skin inflammation, or a pruritus in a subject.

In an additional aspect, the present invention provides a method for treating primary biliary cirrhosis (PBC) in a subject, the method comprising: determining the level of liver alkaline phosphatase (ALP); determining the level of progesterone; and administering a progesterone inhibitor to the subject if the level of ALP or the level of progesterone exceeds a threshold level, thereby treating primary biliary cirrhosis (PBC) in a subject.

In an additional aspect, the present invention provides a method for preventing an itch, a skin inflammation, or a pruritus in a subject, the method comprising: determining the level of liver alkaline phosphatase (ALP); determining the level of progesterone; and administering an agent that antagonizes elevated ALP to the subject if the level of ALP or the level of progesterone exceeds a threshold level, thereby preventing an itch, a skin inflammation, or a pruritus in a subject.

In an additional aspect, the present invention provides a method for treating primary biliary cirrhosis (PBC) in a subject, the method comprising: determining the level of liver alkaline phosphatase (ALP); determining the level of progesterone; and administering an agent that antagonizes elevated ALP to the subject if the level of ALP or the level of progesterone exceeds a threshold level, thereby treating primary biliary cirrhosis (PBC) in a subject.

In another aspect, the invention provides a method for preventing an itch, a skin inflammation, or a pruritus in a subject, wherein said subject is in need of a birth control drug or a progesterone elevating drug, the method comprising: prior to said subject's use of said birth control drug or said progesterone elevating drug, determining the level of a liver enzyme, wherein said liver enzyme is alkaline phosphatase (ALP), alanine transaminase (ALT), or aspartate transaminase (AST); based on the determination of the level of said liver enzyme, determining whether said subject can use said birth control drug or said progesterone elevating drug without the risk of an itch, a skin inflammation, or a pruritus, thereby preventing said itch, said skin inflammation, or said pruritus in said subject.

In another aspect, the invention provides a method for treating an itch, a skin inflammation, or a pruritus in a subject, the method comprising: administering to said subject a progesterone reducing drug, thereby treating said itch, said skin inflammation, or said pruritus in said subject.

In an additional aspect, the present invention provides a method for treating a progesterone related disorder or condition in a female patient, the method comprising testing said patient's levels of ALP, ALT, AST or any combination thereof, and administering a treatment to said patient, wherein said treatment does not elevate progesterone levels if any of ALP, AST or AST levels exceed a predetermined threshold.

In yet additional aspect, the present invention provides a method for determining an appropriate treatment for a progesterone related disorder or condition in a female patient, the method comprising testing said patient's levels of ALP, ALT, AST or any combination thereof, and selecting a treatment that does not elevate progesterone levels if any of ALP, AST or AST levels exceed a predetermined threshold.

In a further aspect, the present invention provides a method for determining an appropriate contraceptive for a female patient, the method comprising testing said patient's levels of ALP, ALT, AST or any combination thereof, and selecting a contraceptive that does not elevate progesterone levels if any of ALP, AST or AST levels exceed a predetermined threshold.

In yet additional aspect, the present invention provides a kit comprising: a progesterone-related contraceptive agent and one or more components for determining the level of ALP, ALT, AST, or progesterone or any combinations thereof, and optionally further comprising an ALP antagonist, progesterone inhibitor, or a combination thereof.

In yet further aspect, the present invention provides a method for treating an itch, a skin inflammation, or a pruritus in a subject having elevated liver alkaline phosphatase (ALP), and further having elevated progesterone, the method comprising: administering to said subject a pharmaceutical composition comprising therapeutically effective amount of a progesterone antagonist.

In yet further aspect, the present invention provides a method for treating an itch, a skin inflammation, or a pruritus in a subject having elevated liver alkaline phosphatase (ALP), and further having elevated progesterone, the method comprising: administering to said subject a pharmaceutical composition comprising therapeutically effective amount of an agent that antagonizes elevated ALP.

Unless otherwise defined, all technical and scientific terms used herein have the same meaning as commonly understood by one of ordinary skill in the art to which this invention belongs. Other features and advantages of the present invention will become apparent from the following detailed description examples and figures. It should be understood, however, that the detailed description and the specific examples while indicating preferred embodiments of the invention are given by way of illustration only, since various changes and modifications within the spirit and scope of the invention will become apparent to those skilled in the art from this detailed description. It is also contemplated that whenever appropriate, any embodiment of the present invention can be combined with one or more other embodiments of the present invention, even though the embodiments are described under different aspects of the present invention.

### DETAILED DESCRIPTION OF THE INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

The term “composition” as used herein is intended to encompass a product comprising specified ingredients in predetermined amounts or proportions, as well as any product which results, directly or indirectly, from combination of the specified ingredients in the specified amounts.

The term “pharmaceutical composition” as used herein is intended to encompass a product comprising one or more active ingredients, and an optional pharmaceutically acceptable carrier comprising inert ingredients, as well as any product which results, directly or indirectly, from combination, complexation or aggregation of any two or more of the ingredients, or from dissociation of one or more of the ingredients, or from other types of reactions or interactions of one or more of the ingredients. In general, pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases. Accordingly, the pharmaceutical compositions of the present invention encompass any composition made by admixing a compound of the present invention and a pharmaceutically acceptable carrier. Said compositions are prepared according to conventional mixing, granulating, or coating methods, respectively, and contain, for example, about 0.1 to 75%, preferably about 1 to 50%, of the active ingredient.

By “pharmaceutically acceptable” it is meant the carrier, diluent or excipient must be compatible with the other ingredients of the formulation and not deleterious to the recipient thereof. Pharmaceutical compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations.

As used herein, “treating” may refer to either therapeutic treatment or prophylactic or preventative measures, wherein the object is to prevent or lessen the targeted pathologic condition or disorder as described hereinabove, or both. Therefore, compositions for use in the methods provided herein may be administered to/contacted with a subject before the onset of diseases, or other conditions described hereinabove. In some cases, compositions for use in the methods provided herein may be administered to/contacted with a subject after the onset of diseases, or other conditions described hereinabove. Thus treating a condition as described herein may refer to preventing, inhibiting, or suppressing the condition in a subject.

Furthermore, as used herein, the terms “treat” and “treatment” refer to therapeutic treatment, as well as prophylactic or preventative measures, wherein the object is to prevent or slow down (lessen) an undesired physiological change associated with a disease or condition. Beneficial or desired clinical results include, but are not limited to, alleviation of symptoms, diminishment of the extent of a disease or condition, stabilization of a disease or condition (i.e., where the disease or condition does not worsen), delay or slowing of the progression of a disease or condition, amelioration or palliation of the disease or condition, and remission (whether partial or total) of the disease or condition, whether detectable or undetectable. “Treatment” can also mean prolonging survival as compared to expected survival if not receiving treatment. Those in need of treatment include those already with the disease or condition as well as those prone to having the disease or condition or those in which the disease or condition is to be prevented. In particular with respect to itch, skin inflammation, or pruritus the terms “treat”, “treating” and “treatment” of chronic pruritus all refer to reducing the frequency of symptoms of acute or chronic pruritus (including eliminating them entirely), avoiding the occurrence of acute or chronic pruritus and/or reducing the severity of symptoms of acute or chronic pruritus.

The terms “administration of” or “administering a” compound should be understood to mean providing a compound of the invention to the individual in need of treatment in a form that can be introduced into that individuals body in a therapeutically useful form and therapeutically effective amount, including, but not limited to, oral dosage forms, such as tablets, capsules, syrups, suspensions, and the like.

The term “therapeutically effective amount” refers to a sufficient quantity of the compounds of the present invention, in a suitable composition, and in a suitable dosage form to treat the noted disease conditions. The “therapeutically effective amount” will vary depending on the compound, the severity of the condition causing the itch, skin inflammation, or pruritus, and the age, weight, etc., of the patient to be treated.

In some embodiments, symptoms are primary, while in other embodiments, symptoms are secondary. As used herein, “primary” refers to a symptom that is a direct result of the disease or disorder, while, “secondary” refers to a symptom that is derived from or consequent to a primary cause. The compositions provided herein may treat primary or secondary symptoms or secondary complications related to the disease or disorder.

As used herein, “symptoms” may be any manifestation of a disease or disorder.

As used herein, “subject” refers to a mammal including a human in need of therapy for, or susceptible to, a condition or its sequelae. The subject may include dogs, cats, pigs, cows, sheep, goats, horses, rats, and mice and humans.

The term “about” or “approximately” means within an acceptable error range for the particular value as determined by one of ordinary skill in the art, which will depend in part on how the value is measured or determined, i.e., the limitations of the measurement system.

The term “inhibitor” as used herein includes compounds that inhibit the expression or activity of a hormone, protein, polypeptide or enzyme and does not necessarily mean complete inhibition of expression and/or activity. Rather, the inhibition includes inhibition of the expression and/or activity of a hormone, protein, polypeptide or enzyme to an extent, and for a time, sufficient to produce the desired effect.

The term “antagonize” as used herein, refers to an act or an effect of decreasing, reducing, or stabilizing the levels of liver alkaline phosphatase (ALP). The desired level may be physiological level of approximately below 200 IU/L. Alternatively, the term may refer to suppression of cyclical fluctuations of ALP, such as, for example, fluctuations dependent on menstrual cycle.

### Pharmaceutical Compositions

Without intending to be bound by any particular mechanism, it is believed that elevated ALP or an underlying condition that leads to elevated ALP (e.g. primary biliary cirrhosis, (PBC) or autoimmune hepatitis (AIH)) in combination with elevated progesterone results in an itch or a pruritus. The rise in progesterone levels may occur naturally and may be either transient (e.g., through cyclical fluctuations related to menstrual cycle) or sustained (e.g., in case of pregnancy). The elevated progesterone levels may also be the result of pharmaceutical intervention, for example the use of progesterone analogs hormonal contraception, or use of progesterone in fertility treatments or prevention of pre-term birth. In addition, the symptoms may appear in the absence of elevated progesterone levels, instead resulting from elevated progesterone sensitivity, such as an autoimmune reaction to the hormone. The resulting itch or pruritus thus may likewise be transient, decreasing in severity or disappearing altogether when progesterone levels decrease (about 9 days after menses), or sustained, as in the course of pregnancy. Pruritus may be otherwise symptom free, or may be accompanied by skin eruption, such as, without limitation, dermatitis, urticaria, papules or plaques. The skin eruptions may be localized to genital area or spread to other areas of the body. The eruptions may resolve at the same approximate time as pruritus following decrease in progesterone levels. While there is no single name for pathologies stemming from the combination of ALD-elevating liver disorders, for example PBC or AIH, and increased progesterone, such pathologies include, without limitation, cyclic vulvovaginitis, vaginal pruritus, autoimmune progesterone dermatitis (APD), or pruritic urticarial papules and plaques of pregnancy (PUPPP)

This invention provides the methods of treating an itch, a skin inflammation, or a pruritus resulting from the above described combination of elevated progesterone (or progesterone hypersensitivity) and a liver condition entailing elevated APD such as, without limitation, PBC or AIH. Such methods can include administering to the subject in need thereof a pharmaceutical composition comprising an effective amount of a progesterone antagonist, an agent that antagonizes elevated ALP, or both, optionally in combination with additional agents.

More specifically, the present invention provides methods for treating an itch, a skin inflammation, or a pruritus in a subject having elevated liver enzyme or elevated progesterone, or both, wherein said liver enzyme is alkaline phosphatase (ALP), alanine transaminase (ALT), aspartate transaminase (AST), or any combination thereof, the method comprising administering to the subject a pharmaceutical composition comprising an effective amount of a progesterone antagonist, an agent that antagonizes elevated ALP, or both, optionally in combination with additional agents.

The term “elevated liver enzyme”, as used herein, may refer to the level of a liver enzyme activity in a patient that is in excess of what is considered normal in the art for a healthy individual. For example, in a healthy female individual the liver alkaline phosphatase (ALP) activity is in the range of 39-177 U/L. Thus a patient whose ALP activity is measured at any value that exceeds the value of about 177 U/L would be considered to have “elevated ALP”. Similarly, in a healthy female individual the liver alanine transaminase (ALT) activity is in the range of 7-55 U/L (see e.g. Mayo Medical Laboratories, http://www. mayomedicallaboratories.com/test-catalog/Clinical+and+Interpretive/8362), and the liver aspartate transaminase (AST) activity is in the range of 8-43 U/L (see e.g. Mayo Medical Laboratories, http://www.mayomedicallaboratories.com/test-catalog/Clinical+and+Interpretive/8360), and a patient whose ALT or AST activity is measured at any value that exceeds the upper limit of these ranges would be considered to have “elevated ALT” or “elevated AST,” respectively.

Suitable agents that antagonize elevated ALP include the agents that treat the underlying cause of elevated ALP, for example primary biliary cirrhosis (PBC) or autoimmune hepatitis (AIP). Such agents include, without limitation, ursodiol, mycophenolic acid, mycophenolate mofetil, vitamin D, chlorambucil, penicillamine, immunosuppressive agents (e.g. cyclosporine, tacrolimus, azathioprine, mycophenolate, and cyclophosphamide), corticosteroids (e.g. budesonide, prednisolone, and predistone), obeticholic acid, mycophenolate, mofetil, thalidomide, methotrexate, malotilate, colchicine, ursodeoxycholic acid, 6-mercaptopurine, and fibrates (e.g. fenofibrate), or pharmaceutically acceptable salts thereof. These agents can be administered alone or in combination.

Suitable progesterone antagonists are inhibitors of progesterone production such as, but not limited to, conjugated estrogen, ethinyl estradiol, danazol, or tamoxifen, or pharmaceutically acceptable salts thereof. In addition agents that block progesterone function, such as, but not limited to mifepristone, stanozolol, ulipristal acetate, lilopristone, or onapristone, or pharmaceutically acceptable salts thereof may be used. Furthermore, general immunosuppressants, such as, without limitation, glucocorticoids, gonadotropin-releasing hormone (GnRH) agonists or alkylated steroids, or pharmaceutically acceptable salts thereof may be used.

Additional suitable compounds for coadministration with the compositions of the present invention are agents that treat the symptoms of PBC and AIP, for example antipruritic agents, which include without limitation rifampin, cholestyramine, opiate antagonists (e.g. naloxone, naltrexone), serotonin antagonists (e.g. ondansetron), antihistamines (e.g. diphenhydramine, loratadine), dronabinol, or pharmaceutically acceptable salts thereof.

Further suitable additional compounds for coadministration with the compositions of the present invention are general sedative agents, such as sertraline.

One or more additional antipruritic agents can optionally be used in combination with the agent that antagonizes elevated ALP and/or progesterone inhibitors to treat said itch, skin inflammation, or pruritus (including acute and chronic pruritus). Antipruritic agents are well known and are fully described, for example, in U.S. Pat. No. 9,381,188, which is incorporated by reference in its entirety. Examples of antipruritic agents include without limitation: antihistamines, including but not limited to antihistamines that inhibit action at the histamine Hi receptor (e.g., acrivastine, antazoline, azelastine, bilastine, brompheniramine, buclizine, bromodiphenhydramine, carbinoxamine, cetirizine, chlorpromazine, cyclizine, chlorpheniramine, chlorodiphenhydramine, clemastine, cyproheptadine, desloratadine, dexbrompheniramine, dexchlorpheniramine, dimenhydrinate, dimetindene, diphenhydramine, doxepin, doxylamine, ebastine, embramine, fexofenadine, hydroxyzine, levocetirizine, loratadine, meclozine, mepyramine, mirtazapine, olopatadine, orphenadrine, phenindamine, pheniramine, phenyltoloxamine, promethazine, pyrilamine, quetiapine, rupatadine, tripelennamine and triprolidine), and antihistamines that inhibit action at the histamine H₄ receptor (e.g., thioperamide, JNJ 7777120 and VUF-6002), and analogs and derivatives thereof; serotonin receptor antagonists, including but not limited to 5-HT₂ antagonists (e.g., clozapine, cyproheptadine, ketanserin, pizotifen and quetiapine) and 5-HT₃ antagonists (e.g., alosetron, cilansetron, dolasetron, granisetron, ondansetron, palonosetron and tropisetron), and analogs and derivatives thereof; neurokinin-1 (NK-1) receptor antagonists, including but not limited to aprepitant, casopitant (GW679769), dapitant, ezlopitant, fosaprepitant, lanepitant (LY-303870), maropitant, netupitant, nolpitant, orvepitant, rolapitant, vestipitant, vofopitant, AV-818, BIIF 1149CL, CP122,721, DNK-333, GSK-424887, L-733060, L-759274, LY-686017, M516102 and TA-5538, and analogs and derivatives thereof; opioid receptor antagonists, including but not limited to butorphanol, cyprodime, levallorphan (lorfan or naloxiphan), nalbuphine, nalorphine (lethidrone or nalline), naloxone, naloxol, nalmefene, naltrexone (e.g., naltrexone 1% cream) and naltrexol, and analogs and derivatives thereof; opioid receptor agonists, including but not limited to selective kappa opioid receptor agonists (e.g., asimadoline, bremazocine, dynorphin, enadoline, ketazocine, nalfurafine, salvinorin A, 2-methoxymethyl salvinorin B, 2-ethoxymethyl salvinorin B, 2-fluoroethoxymethyl salvinorin B, spiradoline, tifluadom, BRL-52537, FE 200665, GR-89696, HZ-2, ICI-199,441, ICI-204,448, LPK-26, U-50488 and U-69,593), and analogs and derivatives thereof; Janus kinase (JAK) inhibitors, including but not limited to JAK1 inhibitors (e.g., GLPG0634 and GSK2586184), JAK2 inhibitors (e.g., lestaurtinib, pacritinib, CYT387 and TG101348), JAK1/JAK2 inhibitors (e.g., baricitinib and ruxolitinib), and JAK3 inhibitors (e.g., tofacitinib), and analogs and derivatives thereof; immunomodulators and immuno suppressants, including but not limited to thalidomide, antimetabolites (e.g., antifolates such as methotrexate), and calcineurin inhibitors (e.g., ciclosporin [cyclosporin], pimecrolimus and tacrolimus), and analogs and derivatives thereof; antidepressants, including but not limited to tricyclic antidepressants (e.g., amitriptyline, amitriptylinoxide, amoxapine, do sulepin [dothiepin], doxepin and melitracen), tetracyclic antidepressants (e.g., amoxapine, maprotiline, mazindol, mianserin, mirtazapine and setiptiline), selective serotonin reuptake inhibitors (SSRIs, e.g., citalopram, dapoxetine, escitalopram, fluoxetine, fluvoxamine, paroxetine and sertraline), and serotonin-norepinephrine reuptake inhibitors (SNRIs, e.g., bicifadine, duloxetine, milnacipran, levomilnacipran, sibutramine, venlafaxine, desvenlafaxine and SEP-227162), and analogs and derivatives thereof; anticonvulsants, including but not limited to carbamazepine, gabapentin, pregabalin, and valproic acid and salts thereof (e.g., sodium valproate), and analogs and derivatives thereof; corticosteroids, including but not limited to hydrocortisone types (e.g., cortisone and derivatives thereof [e.g., cortisone acetate], hydrocortisone and derivatives thereof [e.g., hydrocortisone acetate, hydrocortisone-17-aceponate, hydrocortisone-17-buteprate, hydrocortisone-17-butyrate and hydrocortisone-17-valerate], prednisolone, methylprednisolone and derivatives thereof [e.g., methylprednisolone aceponate], prednisone, and tixocortol and derivatives thereof [e.g., tixocortol pivalate]), betamethasone types (e.g., betamethasone and derivatives thereof [e.g., betamethasone dipropionate, betamethasone sodium phosphate and betamethasone valerate], dexamethasone and derivatives thereof [e.g., dexamethasone sodium phosphate], and fluocortolone and derivatives thereof [e.g., fluocortolone caproate and fluocortolone pivalate]), halogenated steroids (e.g., alclometasone and derivatives thereof [e.g., alclometasone dipropionate], beclometasone and derivatives thereof [e.g., beclometasone dipropionate], clobetasol and derivatives thereof [e.g., clobetasol-17-propionate], clobetasone and derivatives thereof [e.g., clobetasone-17-butyrate], desoximetasone and derivatives thereof [e.g., desoximetasone acetate], diflorasone and derivatives thereof [e.g., diflorasone diacetate], diflucortolone and derivatives thereof [e.g., diflucortolone valerate], fluprednidene and derivatives thereof [e.g., fluprednidene acetate], fluticasone and derivatives thereof [e.g., fluticasone propionate], halobetasol [ulobetasol] and derivatives thereof [e.g., halobetasol proprionate], halometasone and derivatives thereof [e.g., halometasone acetate], and mometasone and derivatives thereof [e.g., mometasone furoate]), acetonides and related substances (e.g., amcinonide, budesonide, ciclesonide, desonide, fluocinonide, fluocinolone acetonide, flurandrenolide [flurandrenolone or fludroxycortide], halcinonide, triamcinolone acetonide and triamcinolone alcohol), and carbonates (e.g., prednicarbate), and analogs and derivatives thereof; local anesthetics, including but not limited to amides (e.g., articaine, bupivacaine, cinchocaine [dibucaine], etidocaine, levobupivacaine, lidocaine [e.g., lidocaine 2.5-5% cream], prilocaine [e.g., prilocaine 2.5% cream], EMLA [lidocaine 2.5%/prilocaine 2.5% cream], mepivacaine, ropivacaine and trimecaine), esters (e.g., benzocaine, chloroprocaine, cocaine, cyclomethycaine, dimethocaine [larocaine], piperocaine, procaine [novocaine], proparacaine, propoxycaine, stovaine and tetracaine [amethocaine]), ethers (e.g., polidocanol [e.g., polidocanol 3% foam] and pramocaine [pramoxine][e.g., pramoxine 1% cream]), and naturally derived local anesthetics (e.g., cocaine, eugenol, menthol, saxitoxin, neosaxitoxin and tetrodotoxin), and analogs and derivatives thereof; counterirritants and cooling agents, including but not limited to capsaicin, camphor, mint oil, menthol (e.g., menthol 1-3% cream), and phenol (e.g., in calamine lotion), and analogs and derivatives thereof; moisturizers, including but not limited to aqueous moisturizers, low pH moisturizers containing an acid (e.g., lactic acid), and moisturizers containing a humectant that attracts and retains water (e.g., glycerol, sorbitol, lactate, urea, and hyaluronic acid and salts thereof), an occlusive that prevents evaporation {e.g., oils (e.g., mineral oil and silicone oil [e.g., dimethicone]) and petroleum jelly (petrolatum)}, and/or an emollient that provides partial hydration and occlusion (e.g., oils, waxes [e.g., lanolin and paraffin], lipids [e.g., phospholipids, ceramides, triglycerides, glycol stearate, glyceryl stearate, fatty acids and squalene], and sterols [e.g., cholesterol and phytosterol]), and analogs and derivatives thereof; and other kinds of antipruritic agents, including but not limited to S-adenosyl methionine, botulinum toxin (e.g., botulinum toxin types A and B), vitamin D and analogs and derivatives thereof (e.g., calcitriol and calcipotriol [calcipotriene]), non-steroidal anti-inflammatory drugs (NSAIDs, e.g., aspirin), cannabinoid receptor agonists (e.g., CB₂ agonists, such as palmitoylethanolamide), inhibitors of cytokines (e.g., antibodies to interleukins, such as IL-31), antagonists of the prostaglandin D₂ receptor (DP₁) and/or the chemoattractant receptor homologous molecule expressed on TH₂ cells (CRTH2) (e.g., TS-022), phosphodiesterase (PDE) inhibitors (e.g., PDE4 inhibitors, such as apremilast), protease-activated receptor 2 (PAR2) antagonists (e.g., GB83), transient receptor potential vanilloid (TRPV) antagonists (e.g., TRPV1 antagonists, such as capsazepine and SB-705498), inhibitors of neurotrophic tyrosine kinase receptors (e.g., TrkA inhibitors, such as CT327), antimicrobials (including antibiotics, antifungals, antivirals and antiparasitics, such as crotamiton and rifampin [rifampicin]), bile absorption-reducing or bile sequestering agents (e.g., ursodeoxycholic acid [ursodiol]), ultraviolet radiation (e.g., ultraviolet A and B), and analogs and derivatives thereof.

If desired (e.g., for relief from itch, skin inflammation, or pruritus during the day), a non-sedating antipruritic agent can be used. For example, second-generation and third-generation antihistamines are designed to be non-sedating, or less sedating than first-generation antihistamines. Non-limiting examples of second-generation and third-generation antihistamines include acrivastine, astemizole, azelastine, bepotastine, bilastine, cetirizine, levocetirizine, ebastine, fexofenadine, ketotifen, levocabastine, loratadine, desloratadine, mizolastine, olopatadine, quifenadine, rupatadine and terfenadine.

In some embodiments, a corticosteroid of moderate or medium potency is used in combination with agent that antagonizes elevated ALP and/or a progesterone inhibitor to treat an itch, skin inflammation, or pruritus-associated condition. Examples of corticosteroids having moderate or medium potency include Groups III, IV and V corticosteroids under the 7-group US classification system and Class II corticosteroids under the 4-class European classification system, including without limitation amcinonide 0.1% (e.g., cream), betamethasone dipropionate 0.05% (e.g., Diprosone.®. cream/ointment), betamethasone valerate 0.1% (e.g., cream/ointment), clobetasone butyrate 0.05% (e.g., Eumovate.®. cream), desonide 0.05% (e.g., Tridesilon.®. cream/ointment and DesOwen.®. cream/ointment), fluocinolone acetonide 0.01-0.2% (e.g., Synalar.®. cream/ointment and Synemol.®. cream), flurandrenolide 0.05% (e.g., Cordran.®. tape), fluticasone propionate 0.005% (e.g., Cutivate.®. ointment), fluticasone propionate 0.05% (e.g., Cutivate.®. cream), halometasone 0.05% (e.g., cream), hydrocortisone butyrate 0.1% (e.g., Locoid.®. cream/ointment), hydrocortisone valerate 0.2% (e.g., Westcort.®. cream/ointment), mometasone furoate 0.1% (e.g., Elocon.®. cream/ointment), triamcinolone acetonide 0.025-0.5% (e.g., Aristocort.®. cream/ointment, Kenacomb.®. cream/ointment, Kenalog.®. cream and Viaderm.®. KC cream/ointment), and triamcinolone diacetate 0.5% (e.g., cream/ointment).

The optional additional antipruritic agent(s) can be administered to a subject suffering from itch, skin inflammation, or pruritus concurrently with (e.g., in the same composition as agent that antagonizes elevated ALP and/or a progesterone inhibitor or in separate compositions) or sequentially to (before or after) administration of agent that antagonizes elevated ALP and/or a progesterone inhibitor. These combinations may further include progesterone inhibitors as described above. Agent that antagonizes elevated ALP and the optional additional progesterone inhibitors and/or antipruritic agent(s) independently can be administered in any suitable mode, including without limitation orally, topically (e.g., dermally/epicutaneously, transdermally, mucosally, transmucosally, intranasally [e.g., by nasal spray or drop], opthalmically [e.g., by eye drop], pulmonarily [e.g., by inhalation], bucally, sublingually, rectally and vaginally), by injection or infusion (e.g., parenterally, including intramuscularly, subcutaneously, intradermally, intravenously/intravascularly, and intrathecally), and by implantation (e.g., subcutaneously and intramuscularly). In some embodiments, an antipruritic agent is administered topically (e.g., dermally) if the itch, skin inflammation, or pruritus is localized, and is administered systemically (e.g., orally or intravenously) if the itch, skin inflammation, or pruritus is widespread (generalized) or has a systemic cause. In certain embodiments, agent that antagonizes elevated ALP and/or the optional progesterone inhibitors and additional antipruritic agent(s) are administered orally. In other embodiments, agent that antagonizes elevated ALP and/or the optional progesterone inhibitors additional antipruritic agent(s) are administered topically (e.g., dermally, mucosally, bucally or sublingually).

Tablets may contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, cornstarch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. A tablet may be made by compressing or molding the active ingredient optionally with one or more pharmaceutically acceptable ingredients. Compressed tablets may be prepared by compressing, in a suitable machine, the active ingredient in a free-flowing form such as a powder or granules, optionally mixed with a binder, lubricant, inert diluent, surface active, or dispensing agent. Molded tablets may be made by molding, in a suitable machine, a mixture of the powdered active ingredient and a suitable carrier moistened with an inert liquid diluent.

Compositions for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil. In particular, a pharmaceutical composition of the present invention may comprise a liquid-filled capsule dosage form in which the active ingredient is in solution in certain combinations of liquid and semi-solid excipients. In one embodiment, the invention is directed to a solution comprising the active agent or a pharmaceutically acceptable salt, solvate or polymorph thereof, and an amphiphilic agent, said amphiphilic agent being a fatty acid ester of glycerol, propylene glycol or sorbitol, as described in U.S. Published Application No. 2010/0209496 (Dakou et al.), which is herein incorporated by reference in its entirety. Preferably, the amphiphilic agent consists essentially of mono- and di-glycerides of C8 to C12 saturated fatty acids and mixtures thereof.

The active ingredient of the present invention may be administered in an oral sustained release formulation. “Sustained release” refers to release of an active agent from a dosage form at a rate effective to achieve a therapeutic amount of the agent, or active metabolite thereof, in the systemic blood circulation over a prolonged period of time relative to that achieved by oral administration of a conventional formulation of the agent. Release of the agent occurs over an extended period of hours, for example, over a period of at least 6 hours, over a period of at least 8 hours, over a period of at least 12 hours, or over a period of at least 24 hours.

Suitable topical formulations and dosage forms include ointments, creams, gels, lotions, pastes, and the like, as described in Remington: The Science and Practice of Pharmacy (21.sup.st Edition, University of the Sciences in Philadelphia, 2005). Ointments are semi-solid preparations that are typically based on petrolatum or other petroleum derivatives. The specific ointment base to be used, as will be appreciated by those skilled in the art, is one that will provide for optimum drug delivery, and, preferably, will provide for other desired characteristics as well, e.g., emolliency or the like. Creams are viscous liquids or semisolid emulsions, either oil-in-water or water-in-oil. Cream bases are water-washable, and contain an oil phase, an emulsifier and an aqueous phase. The oil phase, also called the “internal” phase, is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation is generally a nonionic, anionic, cationic or amphoteric surfactant. Gels are semisolid, suspension-type systems. Single-phase gels contain organic macromolecules (polymers) distributed substantially uniformly throughout the carrier liquid, which is typically aqueous, but also, preferably, contain an alcohol such as ethanol or isopropanol and, optionally, an oil. In order to prepare a uniform gel, dispersing agents such as alcohol or glycerin can be added, or the gelling agent can be dispersed by trituration, mechanical mixing or stirring, or combinations thereof. Lotions are preparations to be applied to the skin surface without friction, and are typically liquid or semiliquid preparations in which solid particles, including the active agent, are present in a water or alcohol base. Lotions are usually suspensions of finely divided solids and will typically contain suspending agents to produce better dispersions as well as compounds useful for localizing and holding the active agent in contact with the skin. Pastes are semisolid dosage forms in which the active agent is suspended in a suitable base. Depending on the nature of the base, pastes are divided between fatty pastes or those made from single-phase aqueous gels.

Various additives, known to those skilled in the art, may be included in the topical formulations. For example, solvents, including relatively small amounts of alcohol, may be used to solubilize certain drug substances. Other optional additives include opacifiers, antioxidants, fragrance, colorant, gelling agents, thickening agents, stabilizers, surfactants and the like. Other agents may also be added, such as antimicrobial agents, to prevent spoilage upon storage, i.e., to inhibit growth of microbes such as yeasts and molds. For those drugs having an unusually low rate of permeation through the skin or mucosal tissue, it may be desirable to include a permeation enhancer in the formulation. The formulation may also contain irritation-mitigating additives to minimize or eliminate the possibility of skin irritation or skin damage resulting from the drug, the enhancer, or other components of the dosage form. The formulations may also contain ether physiologically acceptable excipients or other minor additives, such as fragrances, dyes, emulsifiers, buffers, cooling agents (e.g. menthol), antibiotics, stabilizers or the like. In some instances, one component may serve more than one function.

The concentration of the active agent in a topical formulation can vary a great deal, and will depend on a variety of factors, including the disease or condition to be treated, the nature and activity of the active agent, the desired effect, possible adverse reactions, the ability and speed of the active agent to reach its intended target, and other factors within the particular knowledge of the patient and physician.

In some embodiments, a topical dosage form of agent that antagonizes elevated ALP or inhibits progesterone is formulated as a buccal or sublingual tablet or pill. Advantages of a buccal or sublingual tablet or pill include avoidance of first-pass metabolism and circumvention of gastrointestinal absorption. In addition to a therapeutically effective amount of agent that antagonizes elevated ALP or inhibits progesterone, the buccal or sublingual tablet or pill can contain suitable excipients, including without limitation any combination of fillers and diluents (e.g., mannitol and sorbitol), binding agents (e.g., sodium carbonate), wetting agents (e.g., sodium carbonate), disintegrants (e.g., crospovidone and croscarmellose sodium), lubricants (e.g., silicon dioxide [including colloidal silicon dioxide] and sodium stearyl fumarate), stabilizers (e.g., sodium bicarbonate), flavoring agents (e.g., spearmint flavor), sweetening agents (e.g., sucralose), and coloring agents (e.g., yellow iron oxide). The buccal or sublingual tablet or pill containing agent that antagonizes elevated ALP can be used to treat, e.g., any itch, skin inflammation, or pruritus-associated condition described herein.

The pharmaceutical compositions of the present invention may be formulated as a depot formulation for administration via intramuscular or subcutaneous injection. Depot formulations are efficient, well-tolerated, sustained or delayed release compositions of the active ingredient that are therapeutically effective for a number of weeks, such as at least one week, at least two weeks, at least three weeks, at least four weeks, at least five weeks, or at least six weeks or more. In addition to the active agent, additional ingredients may be used in the depot formulations of the present invention including surfactants, solubilizers, emulsifiers, preservatives, isotonicity agents, dispersing agents, wetting agents, fillers, solvents, buffers, stabilizers, lubricants, and thickening agents. A combination of additional ingredients may also be used. The amount of the active ingredient in a depot formulation will depend upon the severity of the itch, skin inflammation, or pruritus being treated.

The compositions of the present invention may also be presented as a kit, whereby two or more components, which may be active or inactive ingredients, carriers, diluents, and the like, are provided with instructions for preparation of the actual dosage form by the patient or person administering the drug to the patient. Such kits may be provided with all necessary materials and ingredients contained therein, or they may contain instructions for using or making materials or components that must be obtained independently by the patient or person administering the drug to the patient.

The kits of the present invention comprise a contraceptive product (e.g. a progesterone-related contraceptive agent, such as Nuva Ring) and one or more components necessary for testing liver enzyme activity in a patient, for example labeled substrates that are metabolized by liver alkaline phosphatase (ALP), alanine transaminase (ALT), or aspartate transaminase (AST) so as to produce excretable labeled products. The kits of the present invention may further contain one or more components necessary for testing levels of progesterone levels in a patient. The kits of the present invention may also contain an ALP antagonist, a progesterone inhibitor or a combination thereof.

The present invention also contemplates multiple pharmaceutical regimens. Thus the compositions of the present invention may be administered prophylactically, at specific intervals over predetermined period (e.g. pregnancy). In addition the present invention also contemplates preventive administration of the inventive compositions, that is, in the period prior and during the expected symptoms. Finally the present invention contemplates reactive administration of the inventive compositions, that is, in response to the symptoms and other relevant physiological indicators (e.g. menstrual cycling).

In some embodiments of the invention, methods further comprising assessing progression of said itch, skin inflammation, or pruritus in said subject are provided. This assessing step can be performed before said administering step, after said administering step, or combination thereof.

This invention further provides a method for treating a female patient having elevated levels of ALP or an underlying condition that leads to elevated ALP (e.g. primary biliary cirrhosis, (PBC) or autoimmune hepatitis (AIH)). Given that combination of elevated ALP and elevated progesterone results in pruritus, the methods of present invention provide for avoiding treating patients having elevated ALP with pharmaceuticals that elevate progesterone levels either directly or indirectly. For example, according to the present invention, hormonal contraceptives, which include progesterone or its derivatives (e.g. Nuva Ring), are contraindicated to patients having elevated ALP, requiring selection of an alternative contraception method. Non-hormonal contraception methods are well known in the art and include, without limitation, intrauterine devices, mechanical barriers (e.g. diaphragms), or spermicidal agents.

Thus the present invention provides for a method of a treatment for a progesterone related disorder or condition in a female patient, or determining appropriate contraceptives for a female patient having elevated levels of ALP, ALT, or AST, the method comprising testing ALP, ALT, or AST activity in a patient, and, based on the levels of said enzymes, determining and selecting a treatment or a contraceptive that does not elevate progesterone levels if any of ALP, AST or AST levels exceed a predetermined threshold.

The present invention also provides for a method for treating an adverse reaction, such as pruritus, in a female patient having elevated levels of ALP and further having elevated levels of progesterone, the method comprising administering to said subject a pharmaceutical composition comprising therapeutically effective amount of a progesterone antagonist, an agent that antagonizes elevated ALP, or a combination thereof.

The present invention also provides for a method for treating an adverse reaction, such as pruritus, in a female patient, and further having elevated levels of ALP, and receiving progesterone treatment, the method comprising administering to said subject a pharmaceutical composition comprising therapeutically effective amount of a progesterone antagonist, an agent that antagonizes elevated ALP, or a combination thereof.

This invention additionally provides for a method for treating an adverse reaction, such as pruritus, in a female patient having elevated levels of ALP or an underlying condition that leads to elevated ALP (e.g. primary biliary cirrhosis, (PBC) or autoimmune hepatitis (AIH)). Given that the combination of elevated ALP and elevated progesterone results in pruritus, the inventive method provides for testing a patient's ALP levels and avoiding treating patients having ALP levels that exceed a predetermined threshold with pharmaceuticals that elevate progesterone levels either directly or indirectly, thereby avoiding an adverse reaction caused by the combination.

This invention additionally provides for a method for treating an itch, a skin inflammation, or a pruritus in a subject having elevated liver alkaline phosphatase (ALP), and further having elevated progesterone, the method comprising: administering to said subject a pharmaceutical composition comprising therapeutically effective amount of a progesterone antagonist.

This invention additionally provides for a method for treating an itch, a skin inflammation, or a pruritus in a subject having elevated liver alkaline phosphatase (ALP), and further having elevated progesterone, the method comprising: administering to said subject a pharmaceutical composition comprising therapeutically effective amount of an agent that antagonizes elevated ALP.

### Determination of Therapeutic Effectiveness

The effectiveness of compositions of the present invention can be tested in experimental animal models of itch, skin inflammation, or pruritus known to those skilled in the art. For example, various mouse models have been utilized to evaluate treatments for itching. Tsukumo et al. describe a model in which 4-ethoxymethylene-2-phenyl-2-oxazolin-5-one (oxazolone) induces chronic dermatitis with an associated itch response in BALB/c mice that can be used to determine whether an anti-pruritic treatment is effective (J. Pharmacol. Sci., 2010, 113:255-262). Costa et al. report a similar model in which Phoneutria nigriventer spider venom is used as the itch inducer (Vascul. Pharmacol., 2006, 45(4):209-14). Analogously, Ohmura et al. use picrylchloride in NC/Nga mice to stimulate scratching behavior (Eur. J. Pharmacol., 2004; 491:191-194). Essentially, itching is induced in the subject animal with an irritating agent, the test compound or a placebo is administered, and the animal observed under controlled conditions. Scratching behavior is quantified and analyzed using standard statistical techniques. A test compound is considered effective if either continuous or severe scratching is suppressed.

The efficacy of the methods and compositions of the present invention in the treatment of acute and chronic itch, skin inflammation, or pruritus can also optionally be evaluated in human clinical trials conducted under appropriate standards and ethical guidelines as set forth by the U.S. Food and Drug Administration (FDA). After the general safety of a drug is determined in Phase I clinical trials conducted in healthy volunteers, Phase II trials assessing the safety and efficacy of the drug in patients with the condition being treated are conducted. Typically, such trials are double-blinded and placebo-controlled, and may be dose-ranging. Phase III studies gather more information about safety and effectiveness by studying different populations and different dosages and by using the drug in combination with other drugs.

Because amelioration of itch, skin inflammation, or pruritus is subject to a patient's own perceptions, it can be difficult to evaluate with typical clinical endpoints. However, two standardized assessment tools have been created and may be used in clinical trials demonstrating the utility of the present invention. The Visual Analog Scale (VAS) is the most commonly used tool to evaluate the intensity of pruritus (N. Q. Phan et al., Acta Derm. Venereol., 2012; 92:502-507). The VAS is a graphic tool with a 100-mm horizontal line with the left end labeled “no symptom” and the right end labeled “worst imaginable symptom”. The patient is asked to draw a vertical line to indicate the horizontal scale at a point that corresponded to the intensity of the symptom. The length from the left end to the vertical mark made by the patient is measured in millimeters. Separation in one-hundredths is regarded as sufficiently sensitive (R. C. Aitken, Proc. R. Soc. Med., 1969, 62:989-993). The results may be analyzed using standard statistical techniques known to those skilled in the art.

In addition to the VAS, the Dermatology Life Quality Index (DLQI) may be used to evaluate the efficacy of a chronic pruritus treatment. The DLQI, a self-administered general dermatology quality of life questionnaire, was originally developed and published in a dermatology clinic at University Hospital of Wales (A. Y. Finlay and G. K. Khan, Clin. Exper. Derm., 1994, 19:210-216). Independent studies have verified that the DLQI is an easy and efficient method for assessing quality of life in dermatology patients (H. B. Hahn et al., J. Am. Acad. Dermatol., 2001, 45(1):44-8). A current version of the simple, ten-question validated questionnaire, with instructions for use and scoring is available from the School of Medicine, Cardiff University, Wales, UK (world wide web URL dermatology.org.uk/quality/).

The following examples are provided to supplement the prior disclosure and to provide a better understanding of the subject matter described herein. These examples should not be considered to limit the described subject matter. It is understood that the examples and embodiments described herein are for illustrative purposes only and that various modifications or changes in light thereof will be apparent to persons skilled in the art and are to be included within, and can be made without departing from, the true scope of the invention.

### EXAMPLES

### Example 1

A 29-year old, white woman, presented with chronic itching that occurred during menstruation and which began within a few weeks after she restarted using Nuva Ring about a year prior to the visit. The itching was predominantly concentrated on hands, feet, back, and stomach area, and the symptoms disappeared as soon as menstruation began. She previously used Nuva Ring for about three years, followed by approximately 5.5 year hiatus. The medical history and gynecological clinical examination were not unusual, with the exception of elevated liver enzymes having been discovered about two years prior to visit. Initial tests did not reveal any identifiable allergies, leading to the initial hypothesis that the itching was non-dermatologically based. The initial treatment thus comprised short-term moderate amounts of Prednisone, with the regiment starting about the time of re-start of the Nuva Ring use. Approximately two months after the start of Prednisone regimen the patient was diagnosed with an overlap syndrome of Primary Biliary Cholangitis/Cirrhosis (PBC) and Autoimmune Hepatitis (AIH). It was suggested that the etiology was drug-related, with the Nuva Ring drugs being the source. Additional testing revealed that Alkaline Phosphatase (ALP) levels fluctuated synchronously with the patient's menstrual cycle, rising to 1,487 IU/L three days prior to monthly cycle and falling to 1,034 IU/L 9 days after the cycle. New treatment regimen was implemented. Nuva Ring was replaced with a non-hormonal intrauterine device (copper) to enable treatment with immunosuppressive drugs. Additionally steroid drugs, Ursodiol (300 mg three times a day) and mycophenolate mofetil (Cellcept, 500 mg, four times a day), regimen was started in order to reduce AST levels, and continued for 1 year. Finally dietary changes eliminating foods that are rich in zinc and vitamin B12 were effected. A significant reduction of the itching symptoms at the time of menstruation was observed.

### Example 2

A woman experiencing pruritic urticarial papules and plaques of pregnancy (PUPPP) will be administered standard liver enzyme test in order to measure levels of alkaline phosphatase (ALP), alanine transaminase (ALT), and aspartate transaminase (AST). Elevated levels of liver enzymes (e.g. AST above 1000 IU/L) will be indicative of primary biliary cirrhosis (PBC), autoimmune hepatitis (AIP), or both. The patients exhibiting elevated liver enzyme levels will be treated with liver enzyme lowering drugs, such as Ursodiol. The dose, frequency of administration, and duration of treatment will depend on alleviation of PUPP symptoms. The pharmaceutical regimen may be combined with additional measures, such as dietary changes aimed at eliminating zinc and vitamin B12, stimulation of vitamin D production in the skin (via exposure to sunlight) and removal or substitution of medications associated with increased levels of alkaline phosphatase (e.g. antidepressants, anti-inflammatory medication, hormonal drugs, etc.) Further measures may include identifying the underlying condition resulting in elevated ALP levels, and treating this condition.

### Example 3

A woman using progesterone based contraceptive (e.g. Nuva Ring) experiencing pruritic chronic itching occurring during menstruation will undergo standard liver enzyme test in order to measure levels of alkaline phosphatase (ALP), alanine transaminase (ALT), and aspartate transaminase (AST). Elevated levels of liver enzymes (e.g. ALP above 177 U/L) will be indicative of primary biliary cirrhosis (PBC), autoimmune hepatitis (AIP), or both. The patients exhibiting elevated liver enzyme levels will switch to a non-progesterone contraceptive and be treated with liver enzyme lowering drugs, such as Ursodiol and/or Leuprorelin (Lupron). The dose, frequency of administration, and duration of treatment (8 months to 1 year) will depend on menstrual cycling as well as on other aspects, for example alleviation of pruritus symptoms or continued use of contraceptives.

Having described the invention with reference to particular compositions, theories of effectiveness, and the like, it will be apparent to those of skill in the art that it is not intended that the invention be limited by such illustrative embodiments or mechanisms, and that modifications can be made without departing from the scope or spirit of the invention, as defined by the appended claims.

What is claimed is:

1. A method for treating an itch, a skin inflammation, or a pruritus in a subject having elevated liver alkaline phosphatase (ALP), and further having elevated progesterone, the method comprising: administering to said subject a pharmaceutical composition comprising therapeutically effective amount of a progesterone antagonist, an agent that antagonizes elevated ALP, or a combination thereof.

2. The method of claim 1, wherein said progesterone antagonist is a progesterone inhibitor.

3. The method of claim 2, wherein said progesterone inhibitor is conjugated estrogen, ethinyl estradiol, danazol, tamoxifen, mifepristone, stanozolol, ulipristal acetate, lilopristone, or onapristone or pharmaceutically acceptable salts thereof.

4. The method of claim 1, wherein said itch, skin inflammation, or pruritus is associated with an autoimmune progesterone dermatitis (APD).

5. The method of claim 1, wherein the subject further has elevated alanine transaminase (ALT), aspartate transaminase (AST), or both.

6. The method of claim 1, wherein said elevated ALP antagonist is ursodiol.

7. The method of claim 1, wherein said elevated ALP antagonist is mycophenolate mofetil.

8. The method of claim 1, wherein said elevated ALP antagonist is vitamin D, chlorambucil, penicillamine, cyclosporine, tacrolimus, azathioprine, mycophenolate, and cyclophosphamide, budesonide, prednisolone, and predistone, obeticholic acid, mycophenolate, mofetil, thalidomide, methotrexate, malotilate, colchicine, ursodeoxycholic acid, 6-mercaptopurine, or fenofibrate, or pharmaceutically acceptable salts thereof.

9. The method of claim 1, wherein said elevated liver ALP is associated with primary biliary cirrhosis (PBC), autoimmune hepatitis (AIP), or both.

10. The method of claim 1, wherein liver ALP level is above 1000 IU/L 9 days after menstrual cycle.

11. The method of claim 1, wherein the subject is a female subject.

12. The method of claim 1, wherein said elevated progesterone is associated with ovulation.

13. The method of claim 1, wherein said elevated progesterone is associated with pregnancy.

14. The method of claim 13, wherein said pregnancy-associated elevated progesterone results in pruritic urticarial papules and plaques of pregnancy (PUPPP).

15. The method of claim 1, wherein said composition is administered orally, topically, or via injection.

16. The method of claim 1, wherein the method further comprises administering a therapeutically effective amount of another agent that threats an itch, a skin inflammation, or pruritus.

17. A method for treating an itch, a skin inflammation, or a pruritus in a subject having elevated liver ALP, the method comprising: administering to said subject a therapeutically effective amount of a progesterone antagonist, an agent that antagonizes elevated ALP, or a combination thereof.

18. The method of claim 17, wherein said progesterone antagonist is a progesterone inhibitor.

19. The method of claim 18, wherein said progesterone inhibitor is conjugated estrogen, ethinyl estradiol, danazol, tamoxifen, mifepristone, stanozolol, ulipristal acetate, lilopristone, or onapristone or pharmaceutically acceptable salts thereof.

20. The method of claim 17 wherein said itch, skin inflammation, or pruritus is associated with an autoimmune progesterone dermatitis (APD).

21. The method of claim 17, wherein said skin inflammation is vaginal pruritus, chronic vulvovaginal pruritus, or pruritic urticarial papules and plaques of pregnancy (PUPPP).

22. The method of claim 17 wherein the subject further has elevated alanine transaminase (ALT), aspartate transaminase (AST), or both.

23. The method of claim, 17 wherein said elevated ALP antagonist is ursodiol.

24. The method of claim 17, wherein said elevated ALP antagonist is mycophenolate mofetil.

25. The method of claim, 17 wherein said elevated ALP antagonist is vitamin D, chlorambucil, penicillamine, cyclosporine, tacrolimus, azathioprine, mycophenolate, and cyclophosphamide, budesonide, prednisolone, and predistone, obeticholic acid, mycophenolate, mofetil, thalidomide, methotrexate, malotilate, colchicine, ursodeoxycholic acid, 6-mercaptopurine, or fenofibrate, or pharmaceutically acceptable salts thereof.

26. The method of claim 17, wherein said elevated liver ALP is associated with PBC, AIP, or both.

27. The method of claim 17, wherein liver ALP level is above 1000 IU/L 9 days after menstrual cycle.

28. The method of claim 17 wherein said composition is administered orally, topically, or via injection.

29. The method of claim 17, wherein the method further comprises administering a therapeutically effective amount of another agent that threats an itch, a skin inflammation, or pruritus.

30. A method for treating an itch, a skin inflammation, or a pruritus in a subject having elevated progesterone, the method comprising: administering to said subject a pharmaceutical composition comprising therapeutically effective amount of a progesterone antagonist, an agent that antagonizes elevated ALP, or a combination thereof.

31. The method of claim 30, wherein said progesterone antagonist is a progesterone inhibitor.

32. The method of claim 31, wherein said progesterone inhibitor is conjugated estrogen, ethinyl estradiol, danazol, tamoxifen, mifepristone, stanozolol, ulipristal acetate, lilopristone, or onapristone or pharmaceutically acceptable salts thereof.

33. The method of claim 30, wherein said skin inflammation is autoimmune progesterone dermatitis (APD).

34. The method of claim 30, wherein said elevated ALP antagonist is ursodiol.

35. The method of claim 30, wherein said elevated ALP antagonist is mycophenolate mofetil.

36. The method of claim, 30 wherein said elevated ALP antagonist is vitamin D, chlorambucil, penicillamine, cyclosporine, tacrolimus, azathioprine, mycophenolate, and cyclophosphamide, budesonide, prednisolone, and predistone, obeticholic acid, mycophenolate, mofetil, thalidomide, methotrexate, malotilate, colchicine, ursodeoxycholic acid, 6-mercaptopurine, or fenofibrate, or pharmaceutically acceptable salts thereof.

37. The method of claim 30, wherein the subject is a female subject.

38. The method of claim 30, wherein said elevated progesterone is associated with ovulation.

39. The method of claim 30, wherein said elevated progesterone is associated with pregnancy.

40. The method of claim 39, wherein said pregnancy-associated elevated progesterone results in PUPPP.

41. The method of claim 30 wherein said composition is administered orally, topically, or via injection.

42. The method of claim 30, wherein the method further comprises administering a therapeutically effective amount of another agent that threats an itch, a skin inflammation, or pruritus.

43. A method for treating an itch, a skin inflammation, or a pruritus in a subject, the method comprising:
determining the level of liver alkaline phosphatase (ALP);
determining the level of progesterone; and
administering progesterone inhibitor to the subject if the level of a liver enzyme or the level of progesterone exceeds a threshold level
thereby preventing an itch, a skin inflammation, or a pruritus in a subject.

44. A method for treating primary biliary cirrhosis in a subject, the method comprising:
determining the level of liver alkaline phosphatase (ALP);
determining the level of progesterone; and
administering progesterone inhibitor to the subject if the level of a liver enzyme or the level of progesterone exceeds a threshold level
thereby treating primary biliary cirrhosis in a subject.

45. A method for treating an itch, a skin inflammation, or a pruritus in a subject, the method comprising:
determining the level of liver alkaline phosphatase (ALP);
determining the level of progesterone; and
administering an agent that antagonizes elevated ALP to the subject if the level of a liver enzyme or the level of progesterone exceeds a threshold level
thereby preventing an itch, a skin inflammation, or a pruritus in a subject.

46. A method for treating primary biliary cirrhosis in a subject, the method comprising:
determining the level of liver alkaline phosphatase (ALP);
determining the level of progesterone; and
administering an agent that antagonizes elevated ALP to the subject if the level of a liver enzyme or the level of progesterone exceeds a threshold level
thereby treating primary biliary cirrhosis in a subject.

47. A pharmaceutical composition comprising an agent that antagonizes elevated ALP, a progesterone antagonist, or a combination thereof, wherein said agent, antagonist, or both are present in said composition in an amount effective to treat an itch, a skin inflammation, or a pruritus in a subject having elevated liver alkaline phosphatase (ALP), and further having elevated progesterone.

48. A pharmaceutical composition comprising an agent that antagonizes elevated ALP, a progesterone antagonist, or a combination thereof, wherein said agent, antagonist, or both are present in said composition in an amount effective to treat an itch, a skin inflammation, or a pruritus in a subject having elevated liver ALP.

49. A pharmaceutical composition comprising an agent that antagonizes elevated ALP, a progesterone antagonist, or a combination thereof, wherein said agent, antagonist, or both are present in said composition in an amount effective to treat an itch, a skin inflammation, or a pruritus in a subject having elevated progesterone.

50. A method for treating an itch, a skin inflammation, or a pruritus in a subject, wherein said subject is in need of a birth control drug or a progesterone elevating drug, the method comprising:
prior to said subject's use of said birth control drug or said progesterone elevating drug, determining the level of a liver enzyme, wherein said liver enzyme is alkaline phosphatase (ALP), alanine transaminase (ALT), or aspartate transaminase (AST);
based on the determination of the level of said liver enzyme, determining whether said subject can use said birth control drug or said progesterone elevating drug without the risk of an itch, a skin inflammation, or a pruritus,
thereby preventing said itch, said skin inflammation, or said pruritus in said subject.

51. A method for treating an itch, a skin inflammation, or a pruritus in a subject, the method comprising: administering to said subject a progesterone reducing drug, thereby treating said itch, said skin inflammation, or said pruritus in said subject.

52. A method for treating a progesterone related disorder or condition in a female patient, the method comprising testing said patient's levels of ALP, ALT, AST or any combination thereof, and administering a treatment to said patient, wherein said treatment does not elevate progesterone levels if any of ALP, AST or AST levels exceed a predetermined threshold.

53. A method for determining an appropriate treatment for a progesterone related disorder or condition in a female patient, the method comprising testing said patient's levels of ALP, ALT, AST or any combination thereof, and selecting a treatment that does not elevate progesterone levels if any of ALP, AST or AST levels exceed a predetermined threshold.

54. A method for determining an appropriate contraceptive for a female patient, the method comprising testing said patient's levels of ALP, ALT, AST or any combination thereof, and selecting a contraceptive that does not elevate progesterone levels if any of ALP, AST or AST levels exceed a predetermined threshold.

55. A kit comprising a progesterone-related contraceptive agent and one or more components for determining the level of ALP, ALT, AST, or progesterone or any combinations thereof, and optionally further comprising an ALP antagonist, a progesterone inhibitor, or a combination thereof.

56. A method for treating an itch, a skin inflammation, or a pruritus in a subject having elevated liver alkaline phosphatase (ALP), and further having elevated progesterone, the method comprising: administering to said subject a pharmaceutical composition comprising therapeutically effective amount of a progesterone antagonist.

57. A method for treating an itch, a skin inflammation, or a pruritus in a subject having elevated liver alkaline phosphatase (ALP), and further having elevated progesterone, the method comprising: administering to said subject a pharmaceutical composition comprising therapeutically effective amount of an agent that antagonizes elevated ALP.

58. A method for identifying a contraceptive agent for a female subject, the method comprising: determining the level liver alkaline phosphatase (ALP) in said subject; based on the determination of the level of ALP, determining whether said subject is suitable for said contraceptive agent without the risk of an itch, a skin inflammation, or a pruritus, thereby identifying said contraceptive agent for said subject.

59. The method of claim 58, wherein said contraceptive agent is a progesterone elevating contraceptive agent.

60. The method of claim 58, wherein said contraceptive agent is NUVARING.

61. A kit comprising: a progesterone-related contraceptive agent; one or more components for determining the level of liver alkaline phosphatase (ALP), or progesterone or any combinations thereof; and optionally further comprising an ALP antagonist, progesterone inhibitor, or a combination thereof.
